(19) United States
(12) Patent Application Publication
(10) Pub. No.: US 2025/0057166 A1
(43) Pub. Date: Feb. 20, 2025

(54) Method and Apparatus for Disbursement of Ozonated Water

(71) Applicant: Agape Pure, LLC, Rio Rancho, NM (US)

(72) Inventor: Carla Talamantez, Rio Rancho, NM (US)

(73) Assignee: Agape Pure, LLC, Rio Rancho, NM (US)

(21) Appl. No.: 18/219,693

(22) Filed: Aug. 16, 2023

(51) Int. Cl. A01N 59/00; A01P 1/00; A61L 2/22; A61L 2/26

(52) U.S. Cl. CPC A01N 59/00; A01P 1/00; A61L 2/22; A61L 2/26; A61L 2202/11; A61L 2202/16

(57) ABSTRACT

A method for dispersing a stabilized ozonated water solution in a generally uniform manner such that substantially all surfaces within a closed environment are treated with disinfectant/sanitizer. The method of the present invention consists of mixing highly charged air and substantially particulate free water in a constant pressure differential venturi to create ozonated water, then infusing the ozonated water with an inert gas to create a stabilized ozonated water solution, thereby preventing the ozonated water within the solution from returning to the naturally occurring [H₂O] molecular state. The stabilized ozonated water is then stored in a pressurized delivery mechanism such that it may be dispersed in a plurality of forms.

This is a non-provisional application for a utility patent within the meaning of 35 USC 111(a). Priority is claimed based on provisional application US 63/391,728. U.S. Pat. No. 10,792,625 is herein incorporated by reference.

### BRIEF DESCRITION

A method for dispersing a stabilized ozonated water solution in a generally uniform manner such that substantially all surfaces within a closed environment are treated with disinfectant/sanitizer. The method of the present invention consists of mixing highly charged air and substantially particulate free water in a constant pressure differential venturi to create ozonated water, then infusing the ozonated water with an inert gas to create a stabilized ozonated water solution, thereby preventing the ozonated water within the solution from returning to the naturally occurring [H₂O] molecular state. The stabilized ozonated water is then stored in a pressurized delivery mechanism such that it may be dispersed in a plurality of forms.

### BACKGROUND OF THE INVENTION

Production and use of ozonated water has been known in the art for several centuries. First discovered in 1785 by the Dutch chemist Martinus van Marum, it has seen many uses throughout history. For example, as early as 1856 ozonated water has been used as a disinfectant in surgeries. The first documented ozone generator was developed in 1857 by Werner von Siemens, the first report of ozone used to purify blood occurred in 1870 by Dr. C. Lender, and in 1892 it was used as a treatment for tuberculosis.

Contemporarily ozonated water is used to kill viruses, bacteria, algae and fungi. Ozonated water breaks down harmful synthetic chemicals into less dangerous molecules and has been used to purify human blood [see for example the Journal of Ozone Therapy, Vol. 2, No. 2, 2018]. Biochemically, ozone disrupts the integrity of the bacterial cell envelope through oxidation of the phospholipids and lipoproteins. In fungi, ozone inhibits cell growth at certain stages. With viruses, ozone damages the viral capsid and upsets the reproductive cycle by disrupting the virus-to-cell contact with peroxidation. The weak enzyme coatings on cells which make them vulnerable to invasion by viruses also make them susceptible to oxidation and elimination from the body.

While there are numerous methods for producing ozonated water, contemporarily there are three methods in general use: hot spark, ultraviolet light, and cold plasma. In the past, hot spark production, more commonly referred to as coronal discharge, was used mostly for industrial applications, but modernly coronal discharge devices are available for personal applications, allowing economical adaptation for personal and small business use. A functional example is the Tersano iClean Mini, which can be used for cleaning tasks in homes and small businesses.

Ultraviolet and cold plasma are most commonly used for therapeutic work. Cold plasma will produce far greater quantities of ozone in a given time period compared to ultraviolet production. However, that is not to say that the ultraviolet method is not a useful method for producing ozone. When a smaller, steady trickle of ozone is needed, the UV method might be the better choice, for example, for purifying water in a spa or hot tub.

No matter which method for production is used, ozonated water has a very short shelf life. [See for example Characterization Of Water Quality Criteria For Ozonation Processes. Part II: Lifetime of Added Ozone, J. Hoigné & H. Bader, Ozone: Science & Engineering. Vol. 16, Iss. 2, 1994]. Typically, efficacy dissipates in twenty to forty minutes depending on several variables. Note that while the invention disclosed discusses disinfection, it will be understood that sanitization is included as a property of the ozonated water solution created by the invention.

To realize the disinfecting benefits of ozonated water (i.e., a 20 minute efficacy window), the production facility must be collocated, or at least within a short distance of the intended use site. This characteristic of ozonated water is a distinct disadvantage, affecting virtually all contemporary production means. And while the sanitizing efficacy is approximately forty minutes, co-location is still a requirement for contemporary methods.

Recently, U.S. Pat. No. 10,792,625 [Talamantez et al], incorporated herein by reference, disclosed a method for significantly increasing the shelf life of ozonated water, providing for storage in various vessels over time. This major advancement in ozonated water production and distribution leads directly to the use of ozonated water in environments that were previously not economically viable. By way of example, pet care centers that have multiple enclosed spaces for pet boarding, require disinfection many times. As a further example, emergency transport vehicles require disinfection after each patient transfer. The use of a generally uniform atomized mist surface disinfectant solves this problem.

Contemporarily, substitute products such as chlorine in various forms, is used. In particular, chlorine bleach is a heavily corrosive fluid capable of irritating the eyes, skin and respiratory tracts in both animals and humans by simply inhaling the gases its use emits. This inhalation has been demonstrated to deteriorate the lungs and esophagus lining in addition to scarring of the respiratory tract in living entities. Using chlorine bleach as a sanitizer, especially in a pet care setting, can be harmful. As is known, chlorine is also very harmful to the environment since its manufacture produces dioxin.

What would be desirable would be a method for dispersing an ozonated water solution in a generally uniform manner such that all surfaces within a closed environment are treated with disinfectant. What would be further desirable would be a vessel containing an ozonated water solution that could be pre-located and left to operate remotely without human involvement. Even more desirable would be a vessel containing an ozonated water solution under pressure that could be produced at a distance from the use site in an environmentally friendly manner, transported to the use site and stored over a period of time without losing its disinfecting/sanitizing property.

### SUMMARY OF THE INVENTION

A method for dispersing a stabilized ozonated water solution in a generally uniform manner such that substantially all surfaces within a closed environment are treated with disinfectant/sanitizer. The method of the present invention consists of mixing highly charged air and substantially particulate free water in a constant pressure differential venturi to create ozonated water, then infusing the ozonated water with an inert gas to create a stabilized ozonated water solution, thereby preventing the ozonated water within the solution from returning to the naturally occurring [H₂O] molecular state. The stabilized ozonated water is then stored in a pressurized delivery mechanism such that it may be dispersed in a plurality of forms.

Incoming air is charged by exposure to a coronal discharge just prior to entering a venturi. Water is injected into the venturi at a known volume and the pressure across the venturi is held constant. Varying the pressure across the venturi can be used to control the concentration of O₃ in the water.

Downstream from the venturi the ozonated water solution is infused with an inert gas creating a stabilized ozonated water solution. Infusing an inert gas stabilizes the ozonated water thereby preventing a return to the H₂O molecular state. By doing so the shelf life of the disinfecting/sanitizing property of the ozonated water solution is significantly increased. In a preferred embodiment the inert gas is carbon dioxide [CO₂], but as will be recognized by those of skill in the art, other inert gases could be used without departing from the spirit of the invention.

The ozonated water solution from the output of the infusion process is input to a delivery mechanism for end use. Specifically, in a first, preferred embodiment the delivery mechanism is a cannister having a sealing means that can be operated at a future time.

In an alternative embodiment the delivery mechanism is a part of a compact mobile system containing all the required components for producing an ozonated water solution in situ. This embodiment is particularly applicable to such spaces as pet care cubicles, veterinary surgeries and the like.

In operation, the stabilized ozonated water is released into a closed space to be disinfected. Because the stabilized ozonated water is under pressure, it is released in a mist form, allowing substantially all exposed surfaces to be disinfected without the need for a user to physically wipe that surface. Moreover, the multiple embodiments of the present invention provide a means for disinfection of a broad range of spaces to be disinfected.

The method and apparatus of the present invention are discussed below in detail in conjunction with the drawings listed. As will be evident, the method and apparatus of the present invention overcomes the disadvantages of the prior art devices and fulfills a long felt need for extended shelf life of an ozonated water solution. In addition, the apparatus of the present invention provides an efficient means for disinfecting a multitude of closed environments where disinfection of pathogens is needed.

### BRIEF DESCRIPTION OF THE DRAWINGS

FIG. 1: is a block diagram of the method of the present invention.

FIG. 2A: is a schematic presentation of the venturi of the present invention.

FIG. 2B: is a schematic presentation of the container of the present invention.

FIG. 3: is a representation of the disbursement vessel of the present invention.

FIG. 4A: is a block diagram of the compact mobile apparatus of the present invention.

FIG. 4B: is a representation of the compact mobile apparatus of the present invention.

### DETAILED DESCRIPTION OF THE PREFFERRED EMBODIMENTS

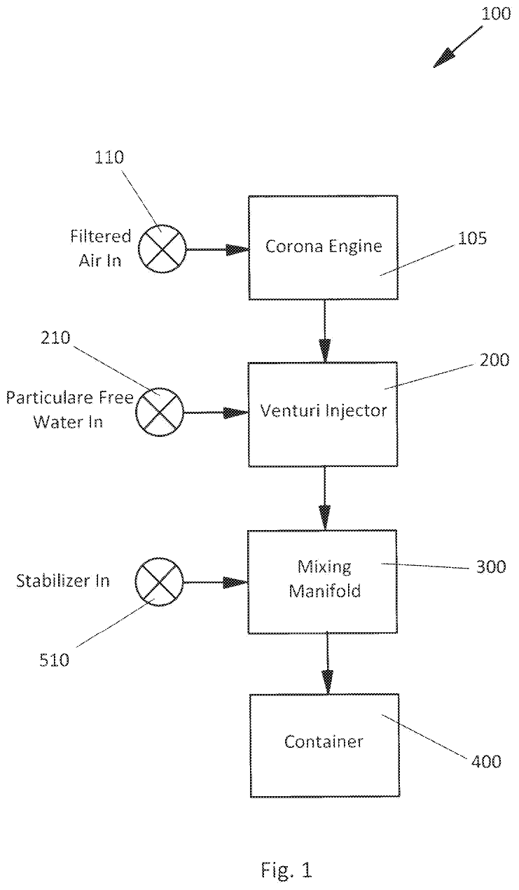

As described briefly above, the method and the plurality of apparatuses of the present invention mitigate the disadvantages of the prior art. Beginning with FIG. 1, a block diagram 100 of the method of the present invention is shown. Corona Engine 105 receives filtered air from Filtered Air In 110. The function of the Corona Engine 105 is to force the incoming air 110 from the single oxygen [O] state to the O₃ state.

The output from the Corona Engine 105 is delivered to the Venturi Injector 200. Particulate Free Water 210 is also delivered to the Venturi Injector 200. The pressure across the venturi is held constant and the ozonated water output of the venturi is delivered to Mixing Manifold 300 where an inert gas Stabilizer In 510 and ozonated water are combined, forming an ozonated water solution. The function of the inert gas is to stabilize the ozonated water solution, thereby maintaining the water in a ozonated state over an extended period of time. The output of the Mixing Manifold 300 is delivered to Container 400 where it is stored under pressure.

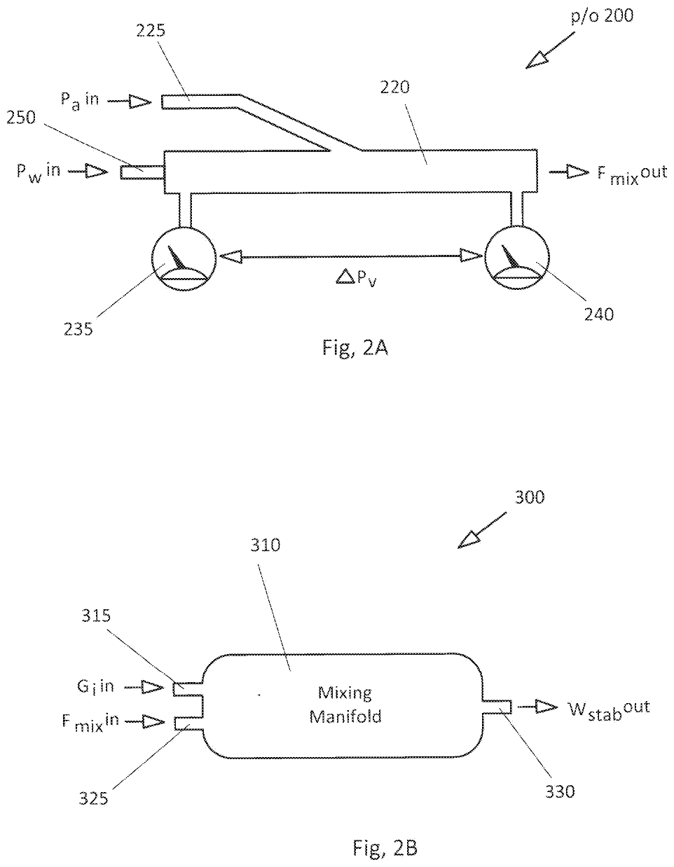

In FIG. 2A a more detailed view of the venturi 200 of the present invention is shown. Operation of venturi 200 is described in detail in U.S. Pat. No. 10,792,625 which has been incorporated by reference herein. By way of a general discussion, however, air Pₐin under pressure enters via tube 225 while particulate free water P_{w}in enters the venturi tube 220 via input connection 250. A constant pressure ΔPᵥ is maintained across the venturi 220 as shown by meters 235 and 240 such that the properly formulated ozonated water Fₘᵢₓout is delivered to the output.

FIG. 2B shows a Mixing Manifold 300 for receiving ozonated water Fₘᵢₓin from the venturi 200 and an inert gas Gᵢin from an external source. Ozonated water Fₘᵢₓin is input to Mixing Manifold 310 via port 325 while inert gas Gᵢin is input via port 315. In one embodiment of the present invention, the inert gas Gᵢin is CO₂, but as is known to those of skill in the art, other inert gases could be used without departing from the spirit of the invention, thus the scope of the invention is limited only by the claims. Within the Mixing Manifold 310 the properly formulated ozonated water is mixed with the inert gas Gᵢin causing the ozonated water to be stabilized. The properly mixed and stabilized ozonated water solution W_{stab}out is output from Mixing Manifold 310 via port 330.

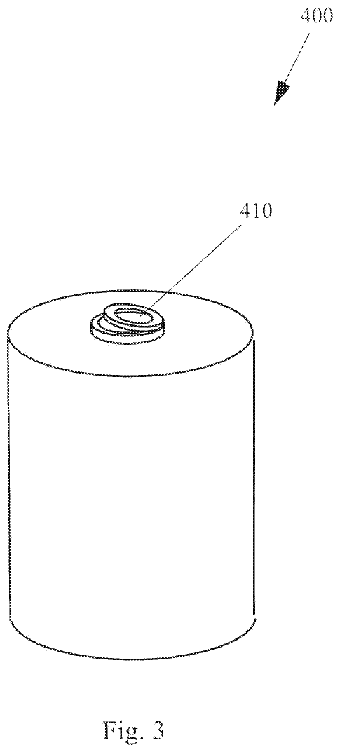

Turning now to FIG. 3, an exemplary container 400 is shown. Container 400 receives the properly mixed and stabilized ozonated water solution W_{stab}out from Mixing Manifold 300 prior to sealing. In this exemplary container, Container 400 is made to accept a snap tab top 410 similar to conventional carbonated drink cans or screw-top bottles. As those familiar with the art will recognize, the seal is accomplished in the conventional manner, thus is not discussed in detail here. In an alternative container, the snap tab top 410 is replaced with a time release valve of the type used on contemporary time release insect foggers as is well known in the art. For example, a pressure regulating valve may be used such that upon activation, the valve regulates the outflow of vapor. It will be obvious to those of skill in the art that other time release valves could be used without departing from the spirit of the invention.

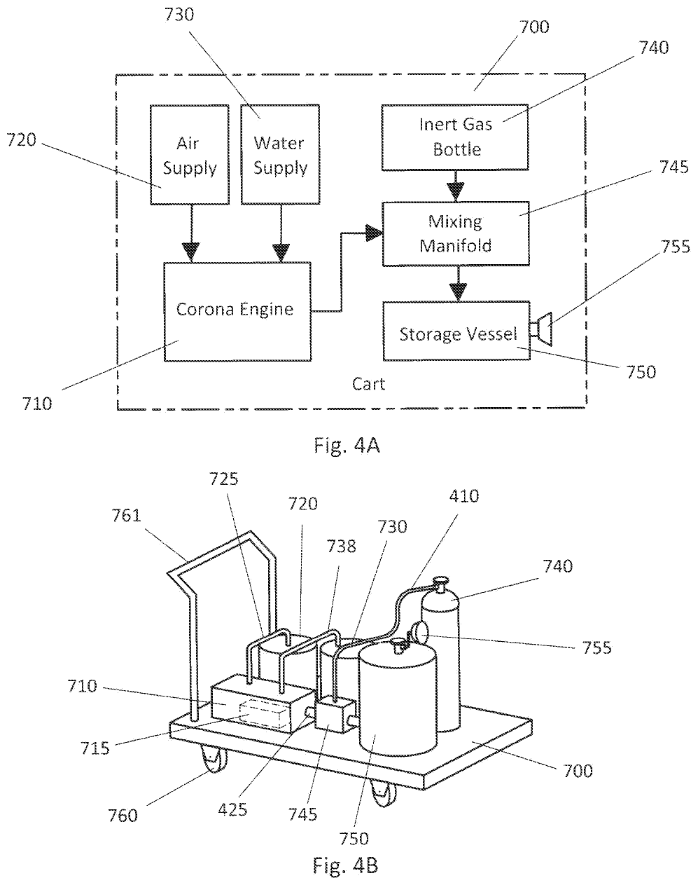

Looking now at FIG. 4, an alternative embodiment of the present invention is shown. In FIG. 4A, a block diagram of a mobile sterilization cart 700 is presented. The mobile sterilization cart 700 is comprised of a mobile cart 700, a Corona Engine 710, an Air Supply 720, a Water Supply 730, an Inert Gas Bottle 740, a Mixing Manifold 745 and a Storage Vessel 750. Of note is the adjustable pattern fogger nozzle 755. Advantageously, the adjustable pattern fogger nozzle 755 may be adjusted to provide an optimum spray pattern for a given enclosed environment. For example, but not meant as a limitation, adjustable pattern fogger nozzle 755 may bet set to a steady stream for a small kennel or pet carrier or to a mist pattern for a veterinary surgery or emergency vehicle interior. Since the pattern is infinitely adjustable, the apparatus of the present invention can address a very wide spectrum of enclosed spaces. As will be recognized, the fogger nozzle 755 could be one of a plurality of nozzle types to accommodate varying environments.

FIG. 4B presents a rendering of the mobile cart apparatus 700. The cart 700 has a flat platform suitable for mounting a corona engine 710, an Air Supply 720, a Water Supply 730, an Inert Gas Bottle 740, a Mixing Manifold 745 and a Storage Vessel 750. The cart 700 is equipped with casters 760 and push bar 761 to allow easy movement of the entire apparatus.

The mobile cart apparatus 700 of FIG. 4 may take a plurality of forms, or configurations. For example, as shown in FIG. 4B, the mobile cart apparatus 700 may be of the “stand-alone” type, wherein a fogger nozzle 755 as described above is used. In another embodiment, mobile cart apparatus 700 may have a length of hose attached to storage vessel 750 [not shown, but well understood in the art]. In this embodiment, the mobile cart apparatus 700 remains stationary while the operator applies storage vessel 750 output to a focused area or surface, for example, a gurney or an operating table.

In operation, Corona Engine 710 receives particulate free water input from Water Supply 730 via water input tube 738 as well as air input from Air Supply 720 via air supply input tube 725. Corona Engine 710 has an internal power source 715 providing the requisite power for the corona engine. Ozonated water output from Corona Engine 710 is delivered to Mixing Manifold 745 via output tube 425. An inert gas from Inert Gas Bottle 740 is delivered to Mixing Manifold 745 via output tube 410. The ozonated water and inert gas are mixed within the Mixing Manifold 745 such that a stabilized ozonated water solution is output to Storage Vessel 750. Adjustable fogger nozzle 755 may now be used to broadcast the stabilized ozonated water solution to an environment to be disinfected.

As can be understood from the foregoing discussion, the method and apparatus of the present invention overcomes many of the problems associated with contemporary disinfection/sterilization of closed environments. And while specific configurations of the present invention have been described in detail, it will be understood by those skilled in the art that these are not the only possible configurations, thus the examples described are exemplary in nature and not meant as a limitation on the scope of the invention.

A first advantage of the present invention is that it provides the capability of dispersing an ozonated water solution in a uniform manner in a variety of closed spaces. This may be accomplished both remotely, through a fogger application, or by a user for more focused application of the ozonated water solution.

A second advantage of the present invention is that the plurality of configurations are self-contained, thus may be used in a very broad spectrum of applications. Various spaces, from very small to room sized may be disinfected using one of the plurality of configurations of the present invention, making it applicable to a substantial segment of the disinfectant user population.

A third advantage of the present invention is that it provides an economical advantage over prior art methods that require generation of ozonated water on location such that it may be used before losing its efficacy.

A fourth advantage of the present invention is that it addresses a broader audience of users. Whether an individual home user or a commercial user, the method and apparatus of the present invention provides a stabilized extended shelf life ozonated water solution on location.

A fifth advantage of the present invention is that it provides a stabilized ozonated water solution with substantially improved shelf life without losing efficacy. This characteristic of the present invention fulfills a long felt need in the disinfectant market.

A sixth advantage of the present invention is that it may be dispersed in a mist form, allowing a plurality of surfaces to be disinfected without the need for physical wiping. This aspect of the present invention allows disinfection without the need for a user to accomplish disinfection through physical action.

A seventh advantage of the present invention is that it does not need continuous power. This is true because the method of the present invention substantially increases the shelf life of the ozonated water solution without losing its disinfecting efficacy.

An eighth advantage of the present invention is that it is non-toxic, allowing for use in medical environments.

What is claimed is:

1. A method for disinfecting a closed space using a stabilized ozonated water solution comprising:
mixing highly charged air and substantially particulate free water in a constant pressure differential venturi to create ozonated water;
infusing said ozonated water with an inert gas in a mixing manifold such that said ozonated water is transformed into a stabilized ozonated water solution, and:
storing said stabilized ozonated water solution in a delivery mechanism under pressure from said inert gas such that said stabilized ozonated water is released in mist form at a future time.

2. The method of claim 1 wherein the inert gas is CO₂.

3. The method of claim 1 wherein the delivery mechanism is a cannister.

4. The cannister of claim 3 wherein said cannister has a time release mechanism.

5. The cannister of claim 3 wherein said cannister has a snap tab top mechanism.

6. The method of claim 1 wherein the delivery mechanism is a self-contained cart.

7. An apparatus for disinfecting a closed space using stabilized ozonated water comprising:
a mobile cart;
an ozonated water generator, said ozonated water generator having an internal source of power, an input source of substantially particulate free water and an input source of pressurized air, the output of said stabilized ozonated water generator being connected to a first input of a mixing manifold:
a pressurized inert gas vessel, said pressurized inert gas vessel being connected to a second input of said mixing manifold, and;
the output of said mixing manifold being under pressure from said inert gas is connected to a delivery vessel such that the contents of said delivery vessel may be dispersed in one of a plurality of forms.
